(19) United States
(12) Patent Application Publication
(10) Pub. No.: US 2022/0143191 A1
(43) Pub. Date: May 12, 2022

(54) ADJUVANT BEVERAGE

(71) Applicant: AZIENDA OSPEDALIERO-UNIVERSITARIA POLICLINICO S. ORSOLA-MALPIGHI-BOLOGNA, Bologna (IT)

(72) Inventors: Rita GOLFIERI, Bologna (IT); Marco STORCHI, Bologna (IT); Alberta CAPPELLI, Bologna (IT); Ferdinando GIANNONE, Bologna (IT); Matteo RENZULLI, Bologna (IT)

(73) Assignee: AZIENDA OSPEDALIERO-UNIVERSITARIA POLICLINICO S. ORSOLA-MALPIGHI-BOLOGNA, Bologna (IT)

(21) Appl. No.: 17/434,964

(22) PCT Filed: Mar. 3, 2020
(86) PCT No.: PCT/IB2020/051793

§ 371 Date: Aug. 30, 2021

(30) Foreign Application Priority Data
Mar. 4, 2019 (IT) 102019000003113

(51) Int. Cl. A61K 47/46; A23L 2/68; A23L 2/60; A23L 2/02; A23L 29/256; A23L 29/30; A23L 29/00; A61K 47/36; A61K 9/00

(52) U.S. Cl. CPC A61K 47/46; A23L 2/68; A23L 2/60; A23L 2/02; A23V 2002/00; A23L 29/37; A23L 29/015; A61K 47/36; A61K 9/0095; A23L 29/256

(57) ABSTRACT

An adjuvant beverage, comprising a water-based mixture comprising water in amounts of 80-85% w/w, salts in amounts of 0.2-0.5% w/w, agar agar, and/or vegetable pectin, and/or starches, in amounts of 0.2%-0.5% w/w, Hibiscus sabdariffa tea, and/or herbal tea with mallow and/or oats, in amounts of 0.3-0.5% w/w, simple carbohydrates in amounts of 15-20% w/w, at least one acidifier in amounts of 0.1-0.3% w/w.

The adjuvant beverage can be used as an oral administration contrast agent for the performance of magnetic resonance imaging of the mesenteric small intestine (Entero-MRI).

### TECHNICAL FIELD OF THE INVENTION

The present invention relates to an adjuvant beverage.

More particularly, the present invention relates to an adjuvant beverage particularly effective as an oral administration contrast agent for the performance of imaging of the mesenteric small intestine, particularly in Magnetic Resonance Imaging.

### DESCRIPTION OF THE PRIOR ART

Magnetic resonance imaging (MRI) of the mesenteric small intestine, also called “Entero-MRI”, is a diagnostic technique that allows viewing and studying the small intestine which, otherwise, would be hard to examine by imaging and difficult to evaluate even with endoscopic techniques, relying exclusively on the use of a video capsule.

In fact, gastro-duodenoscopy manages to scan the stomach and duodenum, while colonoscopy only scans the colon.

Entero-MRI is therefore an innovative method which, using latest-generation Magnetic Resonance equipment, allows all intestinal pathologies, both inflammatory (enteritis) and neoplastic to be evaluated.

In particular, Entero-MRI is the reference technique for the evaluation and follow-up of chronic inflammatory bowel diseases, such as (but not limited to) Crohn's disease and ulcerative rectal colitis.

This type of investigation has now replaced traditional small intestine examination techniques, e.g. with barium (serial small bowel).

In fact, compared to this last-mentioned technique, Entero-MRI does not use radiation, it is better tolerated by the patient, and allows more information content to be obtained.

More in detail, the serial study with barium allows to opacify the intestinal lumen and therefore, to evaluate only the effects that any pathology affecting the wall has on the lumen: in other words, with this technique it is possible to identify only indirect signs of a possible bowel wall pathology. Entero-MRI, on the other hand, allows you to directly view the intestinal wall and its various layers, and therefore to study directly the onset site of any pathologies.

It should also be noted that, unlike in traditional examinations, Entero-MRI also allows the evaluation of the whole area surrounding the intestine, including vessels, lymph nodes, the mesentery and the main organs of the abdominal-pelvic district.

Therefore, thanks to the continuous technical improvements of Magnetic Resonance, some sequences of the Entero-MRI currently allow to also perform a dynamic evaluation of intestinal peristalsis, i.e. the small intestine movement, which in some pathologies is altered. Last, but not least, is the possibility of identifying, through Entero-MRI, any neoplastic conditions of the small intestine.

This is a very important feature of this method, since neoplastic lesions of the small intestine are only rarely (in 10% of cases) symptomatic, and therefore in most cases they would go completely undetected for a long time.

However, the use of the Entero-MRI technique has some drawbacks.

The main limit of Entero-MRI is, to date, the preliminary phase before the actual examination, which consists in the administration of the drug Isocolan® to the patient.

This is a contrast agent for oral administration which is not always well tolerated, as many patients find it unpleasant to swallow.

Furthermore, to obtain the desired efficacy, the intake of considerable quantities in a short period of time is necessary.

This can result in the onset of abdominal pain, and/or abdominal distension, diarrhoea and nausea. Moreover, as reported in its package leaflet, Isocolan® has a number of contraindications, which are listed below:
hypersensitivity to the active ingredients or to any of the excipients;
gastrointestinal perforation;
severe inflammatory bowel disease (such as ulcerative recto-colitis, toxic megacolon, Crohn's disease);
intestine occlusive, sub-occlusive or stenotic episodes, gastric stasis, dynamic ileus, paralytic ileus;
abdominal pain of unknown origin, acute colitis, nausea, vomiting, marked accentuation or reduction of peristalsis, rectal bleeding (the presence of one or more of these signs and symptoms requires adequate in-depth examination by a doctor in order to rule out the presence of pathological conditions that contraindicate the use of laxatives, see previous points);
severe state of dehydration;
children under 8 years of age and weighing less than 20 kg;
generally contraindicated in pregnancy.

In this regard, it is noted that most of the contraindications listed above are themselves conditions for which the performance of an Entero-MRI is indicated.

Consequently, the majority of Entero-MRI scans, being performed for pathologies that contraindicate the use of Isocolan®, should be performed with a different contrast agent.

### OBJECTS OF THE INVENTION

The technical aim of the present invention is to improve the state of the diagnostic methods of intestinal pathologies, or in other related sectors.

Within the scope of this technical aim, it is an object of the present invention to develop a natural, more palatable beverage, alternative to Isocolan®, which can be used for administration as a contrast agent for Entero-MRI or other similar diagnostic techniques.

Another object of the present invention is to make an adjuvant beverage available which allows the contraindications that characterise Isocolan® to be overcome.

This aim and these objects are all achieved by an adjuvant beverage according to the attached claim 1.

The adjuvant beverage, in a preferred embodiment thereof, comprises a water-based mixture comprising water in amounts of 80-85% w/w, salts in amounts of 0.2-0.5% w/w, agar agar, and/or vegetable pectin, and/or starches, in amounts of 0.2-0.5% w/w, Hibiscus sabdariffa tea, and/or herbal tea with mallow and/or oats, in amounts of 0.3-0.5% w/w, simple carbohydrates in amounts of 15-20% w/w, at least one acidifier in amounts of 0.1-0.3% w/w.

Salts can include magnesium sulphate, while simple carbohydrates can include apple juice.

The acidifier can be, for example, lemon juice.

It is a mixture pleasant for the patient to swallow, completely natural in its composition and particularly effective, as a contrast agent, for the performance of diagnostic techniques such as Entero-MRI, or the like.

Dependent claims refer to advantageous embodiments of the invention.

### EMBODIMENTS OF THE INVENTION

The adjuvant beverage according to the invention, in all the embodiments described below, generally comprises a mixture of ingredients which, suitably combined, ensure the same effects and the same osmotic characteristics as Isocolan®, as could be observed in preliminary experimental evaluations.

Furthermore, the beverage does not have the same contraindications, it is more pleasant to the taste and is a completely natural product.

According to one aspect of the invention, the beverage is water based and comprises a mixture of four basic components, namely mucilages, salts, polysaccharides, carbohydrates or simple sugars.

Mucilages have a gently laxative action.

Salts have the effect of osmotic purgatives.

Polysaccharides act as stimulants for intestinal peristalsis.

Finally, carbohydrates or sugars are osmotic laxatives.

The synergistic action of the above four fundamental components, surprisingly, allows to obtain, in the patient, the same effects of Isocolan®, without however having the same contraindications. Isocolan® contains, as active ingredients, macrogol (polyethylene glycol) and sodium and potassium salts, and therefore it is a laxative (purgative) with an osmotic action (it acts by retaining water in the bowel).

This effect determines the distension of the mesenteric small intestine, necessary for the evaluation of its wall as the site of any pathology, by means of imaging methods, e.g. Magnetic Resonance Imaging.

The aforementioned components of Isocolan®, which have the effect of relaxing the bowel loops, in the adjuvant beverage according to the invention are replaced by a combination of natural ingredients.

According to a preferred embodiment of the invention, the ingredients included in the adjuvant beverage are the following:
water, present up to about 80-85% by weight;
agar agar, present up to about 0.2-0.5% by weight;
Hibiscus sabdariffa tea (karkadè) present up to about 0.3-0.5% by weight;
filtered apple juice, present up to about 15-20% by weight;
mannitol, present up to about 0.4-0.8% by weight;
magnesium sulphate, present up to about 0.2-0.5% by weight;
lemon juice, present up to about 0.1%-0.3% by weight.

Water has a hydrating action in the mixture.

More specifically, agar agar is a source of polysaccharides and mucilages.

Agar agar is a gelatinous substance that stimulates bowel movements.

It is a gelling agent of natural origin, derived from red algae: more in detail, it derives from the polysaccharide agarose, which constitutes the supporting structure of the cell walls of some red algae known as Agarophyte, belonging to the class of Rhodophyta; this polysaccharide is released by boiling.

Agar agar is composed of mucilage and carrageenan, which help in the process of converting liquids into gel, without the aid of sugar; agar agar is flavourless.

Hibiscus sabdariffa tea (karkadè) is a source of mucilages, which are soothing and slightly laxative. Although Hibiscus sabdariffa tea may have an astringent action due to the presence of tannins, mucilages, when they come into contact with water, form a gelatinous mass that mechanically helps evacuation, carrying out a gently laxative action.

Magnesium sulphate is a source of salts.

The ingestion of magnesium sulphate increases intestinal and stools hydration, giving them a semi-solid or altogether liquid consistency, which facilitates evacuation.

Finally, mannitol and apple juice are a source of simple carbohydrates/sugars.

Mannitol belongs to the category of hexahydric polyalcohols.

It is a natural remedy used as a mild laxative, especially in children.

When taken orally, mannitol behaves like an osmotic laxative, drawing water into the intestinal lumen and increasing the volume and softness of stools.

The increase in faecal volume is, in turn, a powerful stimulus for intestinal peristalsis.

Mannitol, again taken orally, can be used as an alternative sweetener to traditional sucrose.

Its sweetening power is equal to about 50% of that of cooking sugar, with the advantage of being low-calorie and acariogenic, with a metabolism independent of insulin (a particularly useful feature in case of diabetes); however, its laxative effect limits its use in this respect.

Apple juice also has a sweetening action.

The purpose of lemon juice is to improve the taste and organoleptic properties of the beverage.

Furthermore, lemon juice therefore acts as an acidifier in the beverage.

In fact, lemon juice allows a better management of the consistency of the beverage to be obtained, since in a slightly acidic environment (due to the presence of citric acid in the lemon juice) the agar agar will not totally gel.

Lemon juice also has a preservative action.

For example, and in an embodiment of the invention of particular practical interest, in about 900 ml of beverage in total, the amounts of the above ingredients are approximately:
750 g of water;
3 g of agar agar;
4 g of hibiscus tea;
150 g of filtered apple juice;
5 g of mannitol;
3 g of magnesium sulphate;
2 g of lemon juice.

In another embodiment of the invention, the agar agar could be replaced by vegetable pectin or by starches such as kudzu, corn, rice or the like.

In another embodiment of the invention, the Hibiscus sabdariffa tea (karkade) could be replaced by herbal tea with mallow or oats, or the like.

In yet another embodiment of the invention, filtered apple juice could be replaced by filtered pomegranate, carrot or blueberry juice or the like.

In a further embodiment of the invention, mannitol could be replaced by manna sugar, mannite, D-mannitol or sorbitol or xylitol.

In another embodiment of the invention, magnesium sulphate could be replaced by epsom or English salts or magnesium chloride or magnesium hydroxide or the like.

In another embodiment of the invention, lemon juice could be replaced by the juice of another citrus fruit (lime, mandarin, orange, etc.) or by citric acid or malic acid or the like.

As can be seen from its composition, the beverage according to the invention is an entirely natural product.

It allows the same performance as the traditional contrast agent to be obtained, without however the contraindications of the latter and with better palatability.

The final preparation, in its preferred embodiment, is a slightly sweet (thanks to apple juice and mannitol) refreshing and thirst quenching (thanks to Hibiscus sabdariffa tea and magnesium sulphate) slightly acidic (thanks to lemon juice and Hibiscus sabdariffa tea), pleasantly citrussy (thanks to lemon juice) beverage, with the unmistakable red colour given by Hibiscus sabdariffa tea with a slightly thicker consistency, but not gelatinous (thanks to agar agar), and a slightly mineral aftertaste (thanks to magnesium sulphate).

It is an object of the present invention also the process for the preparation of the adjuvant beverage having the characteristics described above.

In one embodiment, the process comprises a phase of mixing the agar agar with water, and a subsequent phase of bringing the water to a boil, making sure that the agar agar has completely dissolved.

Subsequently, there is a phase of infusing the Hibiscus sabdariffa (karkade) tea for 5 minutes, and then a phase of filtering the obtained mixture.

Subsequently, there is a phase of adding the apple juice, lemon juice, magnesium sulphate and mannitol to the mixture, and allowing it to cool at room temperature or 4° C. before using the product.

Once prepared, the beverage will keep for 48-72 hours at 4° C.

In another embodiment according to the invention, the preparation process comprises the following phases:

hot-solubilise the agar agar, and/or vegetable pectin, and/or starches;

hot-solubilise the Hibiscus sabdariffa (karkadè) tea, and/or mallow and/or oat herbal tea, separately from agar agar;

cold-solubilise the magnesium sulphate and mannitol, or any alternatives to these two components; mix the previously prepared solutions according to the principle of geometric dilutions (i.e. starting from the least concentrated component);

add the apple juice solution, and/or filtered pomegranate, carrots, blueberries juice; add the acidifying solution.

The preparation method is very simple, and it can be easily reproduced on an industrial scale.

As indicated above, an object of the present invention is also the use of the adjuvant beverage, having the characteristics described previously, as an oral contrast agent for performing a magnetic resonance imaging of the mesenteric small intestine (Entero-MRI), or for similar diagnostic techniques.

It has thus been seen how the invention achieves the intended purposes.

The beverage according to the invention constitutes an effective alternative to Isocolan®, e.g. the most common contrast agent used to perform the Entero-MRI. I

n particular, it allows to obtain the same results for the correct preparation/setting up of the aforementioned diagnostic technique, without however featuring the potentially harmful contraindications described above.

Furthermore, the beverage has a much more pleasant taste than Isocolan®, and therefore its intake by the patient is a lot less problematic.

Moreover, even intake in relatively high quantities will not cause after-effects in the patients, it being a completely natural product.

Product preparation is quick and simple, and at a relatively low cost.

The present invention has been described according to preferred embodiments, but equivalent variants are still possible without departing from the scope of the appended claims.

1-9. (canceled)

10. An adjuvant beverage, comprising a water-based mixture comprising:
water in amounts of 80-85% w/w;
salts in amounts of 0.2-0.5% w/w;
agar agar, and/or vegetable pectin, and/or starches, in amounts of 0.2-0.5% w/w;
tea of Hibiscus sabdariffa, and/or herbal tea with mallow and/or oats, in amounts of 0.3-0.5% w/w;
simple carbohydrates in amounts of 15-20% w/w;
at least one acidifier in amounts of 0.1-0.3% w/w;

11. An adjuvant beverage according to claim 10, wherein said salts comprise magnesium sulphate, and/or epsom salts, and/or magnesium chloride, and/or magnesium hydroxide, in amounts of 0.2-0.5% w/w.

12. An adjuvant beverage according to claim 11, wherein said simple carbohydrates comprise filtered apple juice, and/or filtered pomegranate, carrot, blueberry juice, in amounts of 15-20% w/w.

13. An adjuvant beverage according to claim 12, comprising mannitol, and/or manna sugar, and/or mannite, and/or D-mannitol, and/or sorbitol, and/or xylitol, in amounts of 0.4-0.8% w/w.

14. An adjuvant beverage according to claim 13, wherein said acidifier comprises the juice of one or more citrus fruits, and/or citric acid, and/or malic acid.

15. A beverage according to claim 14, wherein 900 ml of beverage contain: 750 g of water, 3 g of agar agar, 4 g of Hibiscus sabdariffa tea, 150 g of apple juice, 5 g of mannitol, 3 g of magnesium sulphate, 2 g of lemon juice.

16. A method for the preparation of the adjuvant beverage ac according to claim 10, wherein comprises the following phases:
mixing water and agar agar, and/or vegetable pectin, and/or starches;
bringing the mixture to the boil;
infusing the Hibiscus sabdariffa tea, and/or mallow and/or oat herbal tea in the mix for 5 minutes;
filtering the mixture;
adding the simple carbohydrates and the acidifier to the mixture;
cooling down the mixture.

17. A method for the preparation of the adjuvant beverage according to claim 10, wherein it comprises the following phases:
hot-solubilise the agar agar, and/or vegetable pectin, and/or starches;
hot-solubilise Hibiscus sabdariffa tea, and/or mallow and/or oat herbal tea, separately from agar agar;
cold solubilise the salts and simple carbohydrates;
mix the previously prepared solutions according to the principle of geometric dilutions;
add the acidifying solution.

18. An adjuvant beverage according to claim 10 for use as an oral administration contrast agent for the performance of magnetic resonance imaging of the mesenteric small intestine (Entero-MRI).
